(19) United States
(12) Patent Application Publication
(10) Pub. No.: US 2008/0240977 A1
(43) Pub. Date: Oct. 2, 2008

(54) Air freshener and the manufacturing method thereof

(76) Inventors: Hsin-Lin Liu, Banciao City (TW); Jingshi Lin, Banciao City (TW)

Correspondence Address: BACON & THOMAS, PLLC, 625 SLATERS LANE, FOURTH FLOOR, ALEXANDRIA, VA, 22314-1176, US

(21) Appl. No.: 11/727,695

(22) Filed: Mar. 28, 2007

(51) Int. Cl. A61L 9/013; A61L 9/04

(52) U.S. Cl. 422/4; 422/123

(57) ABSTRACT

The present invention of “an air freshener and the manufacturing method thereof” relates to the product, which can improve the air quality by means of evaporating itself in the air, particularly for an air freshener, which taking tea as main ingredient.

The air freshener of the present invention is made of tea, which being used as main ingredient thereof, and additive starch together with brown alga glue by means of well-mixing and stirring with water as well as drying by heat. If quantity is small, it can be heated by heater to vaporize natural fragrance for achieving the effect of refreshing the air; If quantity is plenty, it can be filled in the mattress or seat cushion to vaporize natural fragrance for achieving the effect of refreshing the air too.

### BACKGROUND OF THE INVENTION

1. Technical Field

The present invention of “an air freshener and the manufacturing method thereof” relates to the product, which can improve the air quality by means of evaporating itself in the air, particularly for an air freshener, which taking tea as main ingredient.

2. Prior Art

The rapid development of the economy brings some benefits to the people, but creates some issues to be urgently solved, particular the problem of the air pollution. Because the air gradually becomes worse while which is one of the key elements for people to live, seeking the fresh air almost becomes the most desire for everybody accordingly. Therefore, various air freshener have being comprehensively used in the environment of household, office, vehicle, public area and the like; But, the popular air freshener currently in the market seen is chiefly made of chemical material with many additives, such as the air freshener of aerosol spray type or the perfume aromatic of evaporation type.

For example, the Taiwan patent “the air freshening compound and the apparatus” published in the TIPA number of 154507 mainly comprising an evaporator.

In corporation with another mating reagent, the compound can be suppressed for evaporation so as to elongate its service life span to some degree; and, the air freshening compound mainly comprises:
(a): A phosphoric acidic saponification agent has a general formula of [R (OCH2CH2)xOPO3H2] with weight factor of being between 2˜15 and wherein the alkyl phenol R as well as the constant x being between 3˜15;
(b): A Mono-Alkyl-ethers of alkylene-glycols with weight factor of being between 0.1˜15%;
(c): At least one kind of alcohol solvent with weight factor being between 3˜20%: And
(d): One or some ingredients selected from any of the perfume, co-solvent, neutralizer, coloring agent and aqueous group to complement the rest weight factor such that the compound in combination of all foregoing ingredients having weight factor up to 100%.

The other example, the mainland China patent “the electric-heating refreshing fragrant flake and the manufacturing method thereof” of publication number of CN1136962 mainly comprising a substrate with absorption in mixture of attar, solvent and discharging retarder. Said after is extracted from the condiments of jasmine, gardenia, cassia and the like; While said solvent is clean oil and said discharging retarder is option of polyvinyl alcohol or alcoholic varnish such that each content of said condiments, clean oil and polyvinyl alcohol is in ratio of (3˜5): (2˜3): (1˜3) proportion.

However, both of the compounds akin to the air freshener contain chemical material with some additives while such chemical material is not only rare to hardly obtain but also easily produce waste gas and waste water, which will cause environmental pollution if being not well-treated by arbitrarily discharge; Moreover, the feature of said compounds akin to the air freshener made of chemical material easily having poor evaporating effect can readily incur chemical residuals, which will create chronic harm to human body due to accumulation from long term usage.

### SUMMARY OF THE INVENTION

All the foregoing facts proves that the prior arts of the conventional products aforesaid still exist some drawbacks need to be urgently improved; hence it is an urgent issue for the present invention to develop an air freshener made of natural material. In view of such drawbacks of the conventional products, the inventor of the present invention is eager to have innovative improvement on it. Through many years of making extraordinarily painstaking efforts as well as diligent studies, researches and developments, eventually, the “air freshener and the manufacturing method thereof” of the present invention is developed and accomplished successfully.

The main object of the present invention is to provide an air freshener made of natural material such that it having no chronic harm to the human body and no environmental pollution.

For the purpose of fulfilling foregoing object, the air freshener of the present invention is made of tea, which being used as main ingredient thereof, and additive starch together with brown alga glue by means of well-mixing and stirring with water as well as drying by heat; each content of said tea, starch and brown alga glue is in ratio of 100 (tea):5˜20 (starch):0.5˜3 (brown alga glue) by mass proportion as well as the optimal ratio is 100 (tea):8˜15 (starch):0.8˜1.5 (brown alga glue). Moreover, said tea can be any option of Oolong tea, green tea, red tea and the like; said starch can be any option of flour, rice flour, yam powder and the like; While said can be manufactured into powder-shape, granule-shape, flake-shape, pellet-shape or lump-shape.

The manufacturing method of the “air freshener” in the present invention is:
(1) Smashing the tea and sieving which via screen of □200 meshes such that becoming powder-shaped tea.
(2) Adding starch and brown alga glue into and mixing with said tea powder, then adding water thereat to mix and stir which such becoming well-mixed in even manner.
(3) Drying said well-mixed tea powder, starch and brown alga glue by heat, then shaping which into powder product, or shaping which into granule product, or shaping which into pellet, flake and lump products.

The air freshener of the present invention is made of natural material, which being the main ingredient thereof, has natural fragrance with more pure odor, more durable smell, no toxicant and harmless feature in conformity with the Act and Regulations of Green Environmental Protection comparing to the existing conventional air freshener, perfume, condiments and refreshing fragrant flake akin, which has strong and stinky odor; Thereby, the air freshener of the present invention not only has feature of neatness, elegance, delight and fad appeal, mind-sobering and strength-reinvigorating sense, but also has advantages of easiness of obtaining ingredients, simplicity of processing and low cost; Thus, it can be used in any space of room, vehicle, public area and the other location, particularly on the heater so that the natural tea scent is evaporated immediately to achieve the air refreshing effect.

When the air freshener of the present invention is specially made into granule with size between 5 m/m˜50 m/m for being put into the mattress or seat mat; Because of the supper absorbability, good sweat absorption and excellent air permeability thereof together with the active factor in the natural poly-phenol of tea evaporated, the air freshener can effectively eliminate the moisture and strange odors and result in eliminating weary sense and improving sleeping quality as well as preventing from a variety of diseases owing to enhancing the blood circulation and metabolism of the brain thereby.

When the air freshener of the present invention is made into granule for serving as the filler of the pillow; Because of not only the particular porous structure and excellent absorptivity but also the ability of the air permeability with neat comfort and the sweat-absorbing with eliminating-moisture as well as the de-odor effect thereof together with the active factor in the natural poly-phenol of tea evaporated, the air freshener can effectively improve the air quality and keep air in fresh and natural condition so that it can overall alleviate body weary sense and improve sleeping quality owing to enhancing the blood circulation and metabolism of the brain thereby.

When the air freshener of the present invention is made into granule for serving as the back cushion or the filler of the bolster; the active factor in the natural poly-phenol of tea evaporated from the air freshener can effectively eliminate the strange odor of formaldehyde to purify and keep the air in fresh and healthy condition wherein so that it can enhance the safety of the vehicle driver, particularly for the new car.

When the air freshener of the present invention is used in the inner space of the room or vehicle, it can be made into flake with weight of being less than 7 g; the active factor in the natural poly-phenol of tea evaporated from heating the air freshener can effectively not only eliminate the strange odor in the room or vehicle to improve the air quality but also have potential to reinvigorate the spirit, sober up the brain, calm down the emotion, soothe mind pressure and suppress the proliferation of the fungus, bacteria, virus and mites as well as prevent from diseases.

When the air freshener of the present invention is made into scent bag, it can not only effectively absorb the strange order in the air to improve the air quality, but also effectively eliminate the harmful gas of formaldehyde and the like evaporating from the furniture so that it can manifest the deodorization and de-mold effect.

### Detailed Description of the Preferred Embodiments

In order to let your honorable examiners understand the contents and the achievable effects of the present invention, the details are described below in coordinated with the accompanying drawing:

Please refer to the manufacturing steps in the FIG. 1, the ingredients in the “air freshener and the manufacturing method thereof” of the present invention are tea, which is main material, and additive starch as well as brown alga glue; the final product of which is obtained by means of mixing, stirring with water and drying by heat.

The steps of the manufacturing method are as below:
(1) Smashing 10: Smashing the tea and sieving which via screen of □200 meshes such that becoming powder-shaped tea.
(2) Mixing 20: Adding starch and brown alga glue into and mixing with said tea powder, then adding water thereat to mix and stir which such becoming well-mixed in even manner.
(3) Shaping 30: Drying said well-mixed tea powder, starch and brown alga glue by heat, then shaping which into powder product, or shaping which into granule product, or shaping which into pellet, flake and lump products.

The selected main ingredient in the first exemplary embodiment of the present invention is the Iron-Kwang-In tea of high quality in weight of 100 kg for being smashed and sieved via screen of □200 meshes such that becoming tea powder; after mingling in the agitating machine for 15 minutes by adding high gluten flour of 10 kg and glue of brown alga of 1 kg, re-stir the mixture of which by adding distilled water of 11 kg, then make which into granule of 10 mm size by powder making machine after re-agitating which into well-mixed manner; further, press said granular mixture in the chip-presser such that becoming flake-shape of 40 mm (length)×25 mm (width)×4 mm (thickness), dry which by heat in the drying apparatus into mixture with water content being less than 5% of total material weight, the air freshener of flake-shape is obtained immediately.

If said air freshener is on the heater of the vehicle, the fragrance of which will be full of all the internal space of the vehicle so that all the vehicle passengers will smell the natural tea fragrance therefrom; Or, said air freshener can be heated by the ceramics heater when it is used in the household, office or public location environments and the other places, the air-refreshing effect will be further achieved as the people in such area will smell the natural tea fragrance therefrom too.

The selected main ingredient in the second exemplary embodiment of the present invention is the Lung-Xin tea from Hang-Zoh county or the Low-Chun tea from Su-Zoh county in mainland China of high quality in weight of 100 kg for being smashed and sieved via screen of □250 meshes such that becoming tea powder; after mingling in the agitating machine for 10 minutes by adding tap-water of 10 kg such that being well-mixed manner; dry which by heat in the drying apparatus into mixture with water content being only 4% of total material weight, the air freshener of powder-shape is obtained immediately; or get the air freshener of pellet-shape, flake-shape or lump-shape by virtue of shaping machine.

The selected main ingredient in the third exemplary embodiment of the present invention is the rock tea from Wu-I mountain in mainland China of high quality in weight of 100 kg for being smashed and sieved via screen of 200 meshes such that becoming tea powder; after mingling in the agitating machine for 20 minutes by adding sweet potato powder of 15 kg and glue of brown alga of 1.5 kg, re-stir the mixture of which by adding clean water, then make which into pellet-like thing of 2˜5 mm size by pellet making machine after re-agitating which into well-mixed manner; then, dry said mixture of which by heat in the drying apparatus into mixture with water content being only 4.5% of total material weight, the air freshener of pellet-shape is obtained immediately.

The selected main ingredient in the fourth exemplary embodiment of the present invention is the green tea of high quality in weight of 100 kg for being smashed and sieved via screen of 250 meshes such that becoming tea powder; after mingling in the agitating machine for 18 minutes by adding sweet corn powder of 5 kg and glue of brown alga of 0.8 kg, re-stir the mixture of which by adding tap-water such that being well-mixed manner; Finally, press said mixture in the chip-presser such that becoming flake-shape of 50 mm (length)×30 mm (width)×4.5 mm (thickness), dry which by heat in the drying apparatus into mixture with water content being less than 4.2%˜4.5% of total material weight, the air freshener of flake-shape is obtained immediately.

The selected main ingredient in the fifth exemplary embodiment of the present invention is the red tea of high quality in weight of 100 kg for being smashed and sieved via screen of 200 meshes such that becoming tea powder;

after mingling in the agitating machine for 18 minutes by adding flour of 12 kg and glue of brown alga of 0.5 kg, re-stir the mixture of which by adding tap-water such that being well-mixed manner;
after pressing said mixture in the chip-presser such that becoming lump-shape of 40 mm (length)×25 mm (width)×10 mm (thickness), dry which by heat in the drying apparatus into mixture with water content being only 4% of total material weight, the air freshener of lump-shape is obtained immediately.

The selected main ingredient in the sixth exemplary embodiment of the present invention is the Oolong tea of high quality in weight of 100 kg for being smashed and sieved via screen of 200 meshes such that becoming tea powder;

after mingling in the agitating machine for 15 minutes by adding tapioca powder of 20 kg and glue of brown alga of 3 kg, re-stir the mixture of which by adding distilled water such that being well-mixed manner;
after pressing said mixture in the chip-presser such that becoming flake-shape of 40 mm (length)×25 mm (width)×4 mm (thickness), dry which by heat in the drying apparatus into mixture with water content being only 4% of total material weight, the air freshener of flake-shape is obtained immediately.

The air freshener in the seventh exemplary embodiment of the present invention is specially made into granule with size between 5 m/m˜50 m/m for being put into the mattress or seat mat; Because of the supper absorbability, good sweat absorption and excellent air permeability thereof together with the active factor in the natural poly-phenol of tea evaporated, the air freshener can effectively eliminate the moisture and strange odors and result in eliminating weary sense and improving sleeping quality as well as preventing from a variety of diseases owing to enhancing the blood circulation and metabolism of the brain thereby.

The air freshener in the eighth exemplary embodiment of the present invention is made into granule for serving as the filler of the pillow; Because of not only the particular porous structure and excellent absorptivity but also the ability of the air permeability with neat comfort and the sweat-absorbing with eliminating-moisture as well as the de-odor effect thereof together with the active factor in the natural poly-phenol of tea evaporated, the air freshener can effectively improve the air quality and keep air in fresh and natural condition so that it can overall alleviate body weary sense and improve sleeping quality owing to enhancing the blood circulation and metabolism of the brain thereby.

The air freshener in the ninth exemplary embodiment of the present invention is made into granule for serving as the back cushion or the filler of the bolster; the active factor in the natural poly-phenol of tea evaporated from the air freshener can effectively eliminate the strange odor of formaldehyde to purify and keep the air in fresh and healthy condition wherein so that it can enhance the safety of the vehicle driver, particularly for the new car.

The air freshener in the tenth exemplary embodiment of the present invention for being used in the inner space of the room or vehicle is made into flake with thickness of 5 m/m and weight of being less than 7 g; the active factor in the natural poly-phenol of tea evaporated from heating the air freshener can effectively not only eliminate the strange odor in the room or vehicle to improve the air quality but also have potential to reinvigorate the spirit, sober up the brain, calm down the emotion, soothe mind pressure and suppress the proliferation of the fungus, bacteria, virus and mites as well as prevent from diseases.

However, all the terms and glossaries are adopted and defined for convenience in describing and disclosing the technological content of the present invention, it is not intended for confining and limiting the range and scope of the claims in the present invention; therefore, any equivalent exchange, substitution or adaptation of the corresponding component or material in accordance with the spirit, nature and essence of the present invention is deemed being included in the scope and encompass of the protection under the claims of the present invention.

### BRIEF DESCRIPTION OF THE DRAWING

FIG. 1 is the manufacturing steps of the present invention.

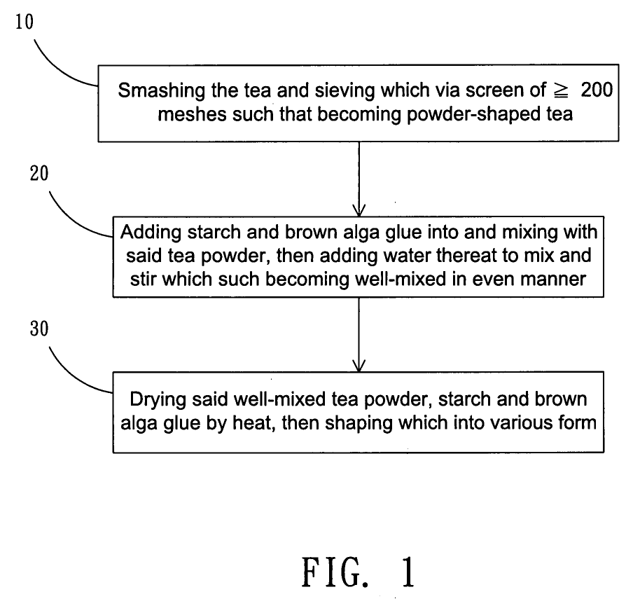

What is claimed is:

1. An “air freshener” is made of tea, which being used as main ingredient thereof, and additive starch together with brown alga glue by means of well-mixing and stirring with water as well as drying by heat, wherein, each content of said tea, starch and brown alga glue is in ratio of 100 (tea):5˜20 (starch):0.5˜3 (brown alga glue) by mass proportion.

2. A manufacturing method of the “air freshener” comprises the steps as below:
(1) Smashing: Smashing the tea and sieving which via screen meshes such that becoming powder-shaped tea.
(2) Mixing: Adding starch and brown alga glue into and mixing with said tea powder, then adding water thereat to mix and stir which such becoming well-mixed in even manner.
(3) Shaping: Drying said well-mixed tea powder, starch and brown alga glue by heat, then shaping which into powder product, or shaping which into granule product, or shaping which into pellet, flake and lump products.

3. The “air freshener”, which being recited in the claim 2, is further manufactured into pellet-shape or lump-shape with thickness of 5 m/m˜50 m/m to serve as the filler of pillow, hugging pillow, back cushion, seat mat or mattress and the like, or into flake-shape with thickness of 5 m/m and weight of being less than 7 g such that it being able to be put on the heater.
